(19) United States
(12) Patent Application Publication
(10) Pub. No.: US 2009/0093775 A1
(43) Pub. Date: Apr. 9, 2009

(54) Microstructures

(76) Inventors: Remesh S/O Govinda Raju, Singapore (SG); Patricia A. Beck, Palo Alto, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/906,883

(22) Filed: Oct. 3, 2007

(51) Int. Cl. A61M 5/158

(52) U.S. Cl. 604/272

(57) ABSTRACT

A microstructure array includes at least one microneedle and at least one ancillary microstructure on or adjacent to the microneedle configured to modulate the function of the microneedle.

### BACKGROUND

Hollow microneedles have been developed which offer utility for delivering drugs or the withdrawal of bodily fluids across biological barriers, such as skin. A microneedle is typically a miniature needle with a penetration depth of about 50-150 μm. The microneedle is typically designed to penetrate the skin, but to avoid striking nerves, thereby avoiding the pain of a conventional injection. An array of microneedles may be combined with an analyte measurement system to provide a minimally invasive fluid retrieval and analyte sensing system. In other fields, solid microneedles are desirable as probes to sense electrical signals or to apply stimulation electrical signals, and hollow microneedles are useful as means for dispensing and/or obtaining small volumes of materials.

A variety of ancillary microstructures have been developed which confer greater utility and reliability on microneedles and/or microneedle arrays, thereby enhancing their function. These structures may be prepared utilizing the same or similar processes employed to fabricate microneedles, and may even be co-fabricated with the microneedles, thereby conferring the advantages of both low manufacturing cost and high manufacturing volume.

### BRIEF DESCRIPTION OF THE DRAWINGS

FIG. 1 is a flow chart illustrating an embodiment of a method for fabricating microstructures.

FIG. 2 is a schematic flow diagram showing a process of fabricating an embodiment of a microneedle according to an embodiment of the method of FIG. 1.

FIG. 3 is an isometric view of a microneedle prepared by the process of FIG. 2.

FIG. 4 is a flow chart illustrating an embodiment of a method for fabricating representative ancillary microstructures.

FIG. 5 is an isometric depiction of an embodiment of an array of selected microstructures.

FIG. 6 is a schematic flow diagram showing an embodiment of a process for fabricating a representative microspring structure.

FIG. 7 is a schematic diagram showing an embodiment of a microspring structure stretching a membrane.

FIG. 8 is a schematic diagram showing an embodiment of a process for fabricating a representative microvalve microstructure.

FIG. 9 is a schematic flow diagram showing an embodiment of a process for fabricating a representative ridged microstructure.

FIG. 10 is a schematic diagram showing a cross-sectional view of an embodiment of a ridged microstructure embedded in a substrate.

FIG. 11 is a schematic diagram showing a cross-sectional view of an embodiment of a microstructure including a flap valve, including (A) a side view, (B) a front view, and (C) a cross-sectional view of the microstructure embedded in a substrate.

FIG. 12 is a schematic diagram showing a cross-sectional view of an embodiment of a microstructure including a barbed structure.

FIG. 13 is a schematic diagram showing a cross-sectional view of a portion of an embodiment of a microstructure array that includes diverting structure.

### DETAILED DESCRIPTION

The present disclosure relates generally to microstructures, and in particular to ancillary microstructures for improving the function of an associated operative microstructure. A microstructure that is on, adjacent to, or in sufficient proximity with an additional operative microstructure to effect its operation may be considered to be associated with that microstructure. In particular, an associated microstructure may be considered an ancillary microstructure where the microstructure is configured to improve or alter the performance characteristics of the operative microstructure.

A non-limiting example of an operative microstructure is a microneedle, or a microneedle array. A microstructure that is on, adjacent to, or in sufficient proximity with a microneedle to effect its operation may be considered to be associated with that microneedle. The associated microstructure may be considered an ancillary microstructure where the microstructure is configured to improve or alter the performance characteristics of that microneedle. In one embodiment, an ancillary microstructure is a microstructure configured to modulate the ability of the microneedle to inject and/or retrieve a selected substance.

An ancillary structure may enhance the operation of an associated microneedle. For example, the presence of an ancillary microstructure may improve the penetration of an associated microneedle through skin or other membrane or target surface. An ancillary microstructure may help increase, decrease, or otherwise meter the flow of a selected substance that is injectable or collectable by an associated microneedle. An ancillary microstructure may be configured to stabilize an associated microneedle during penetration into and/or withdrawal from skin or other membrane or surface, and/or to secure an associated microneedle more firmly into an injection and/or collection site, among other effects.

A variety of methods have been developed for fabricating microstructures. Some methods are described in U.S. Pat. No. 7,097,776 to Raju; U.S. patent application Ser. No. 11/485,504 filed Jul. 12, 2006 and entitled “Microfabrication Method”; and U.S. patent application Ser. No. 11/420,764 filed May 29, 2006 (U.S. Patent Publication No. 2006/0226016) and entitled “Microneedles and Method of Fabricating Thereof”, each of which is incorporated herein by reference for all purposes. As a non-limiting example, microstructures, such as microneedles, may be manufactured from stainless steel and other conductive materials by a plating process that lends itself to low cost, high volume, manufacturing-friendly microfabrication.

These methods utilize a reusable substrate, and permit the fabrication of structure layers having a fully contoured surface, where the surface may incorporate through holes or surface channels anywhere in the structure layer. The structure layer may be prepared with various geometries, and may include controlled breakpoints, self-aligned structures, and other features. In addition, the thickness of the structure layer may also be easily varied, leading to easily created yet robust features. All of this can be accomplished on a reusable substrate using electroplating and/or other fabrication techniques. The ability to co-fabricate a variety of structures on a single reusable substrate significantly increases the reliability of the resulting structure, as well as lowering the overall cost of fabrication.

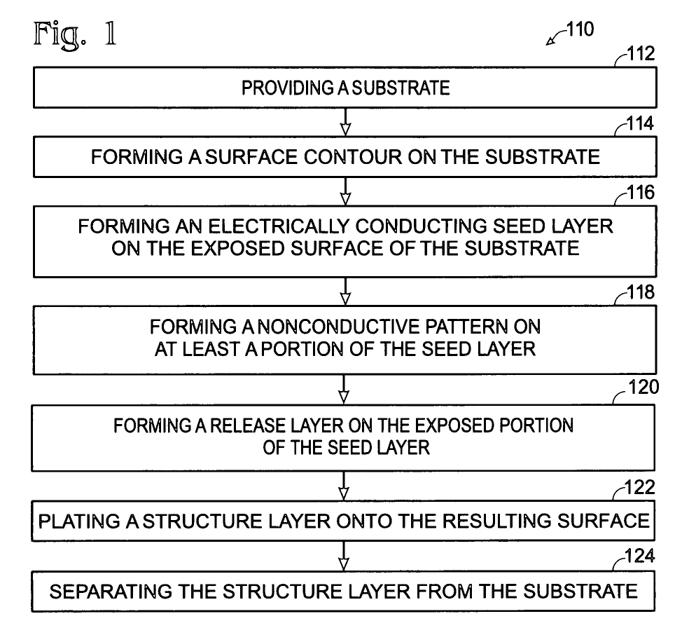

One embodiment of forming a microstructure (one non-limiting example of which may be a microneedle) is depicted in the flowchart 110 of FIG. 1, and includes providing a substrate at 112; forming a surface contour on the substrate at 114; forming an electrically conductive seed layer on the exposed surface of the substrate at 116; forming a nonconductive pattern on at least a portion of the seed layer at 118; forming a release layer on the exposed portions of the seed layer at 120; plating a microneedle structure layer onto the resulting surface to create the desired microneedle structure at 122; and separating the microneedle structure layer from the substrate at 124.

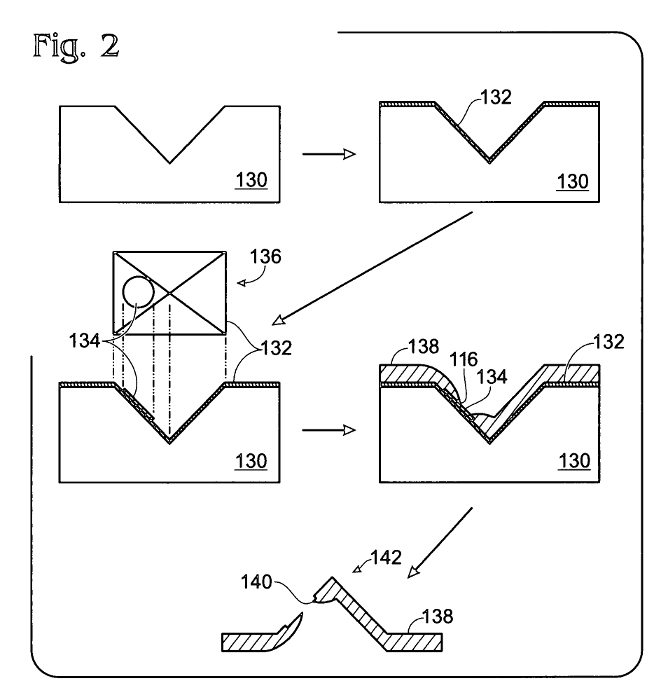

One embodiment of a microfabrication process for a microneedle is depicted schematically in FIG. 2, beginning with the preparation of a substrate 130 in which is formed the desired surface contour. A seed layer 132 is then applied over this contour. In this embodiment, the seed layer 132 acts as a release layer. It is to be understood, however, that a separate release layer may be established (as discussed further herein). In FIG. 2, a non-conductive pattern 134 is subsequently formed over a portion of this seed layer. This nonconductive pattern assists in controlling the manner in which the electroplated layer grows on surface of the substrate 130. Thus, by varying the dimensions of the non-conductive region(s) and the thickness of the plated materials, the microneedle structure can be created. A top view of the seed layer 132 and the patterned non-conductive layer 134 is shown at 136.

An exterior contour of a microneedle structure 138 is electroplated onto the substrate 130, following the shape of the concavity in the substrate 130. An aperture 140 remains in the electroplated layer due to the presence of nonconductive layer 134. The electroplated microneedle 142 may then be separated from the seed layer 132 [e.g., via the heating of a release layer (which, as previously described, may or may not be a separate layer from the seed layer 132) or some other suitable method]. The fabricated microneedle 142 is shown in FIG. 3. The microneedle is configured to be inserted into skin or other membrane and/or surface, for example, and void 140 permits distribution or collection of material, such as the injection of medicament.

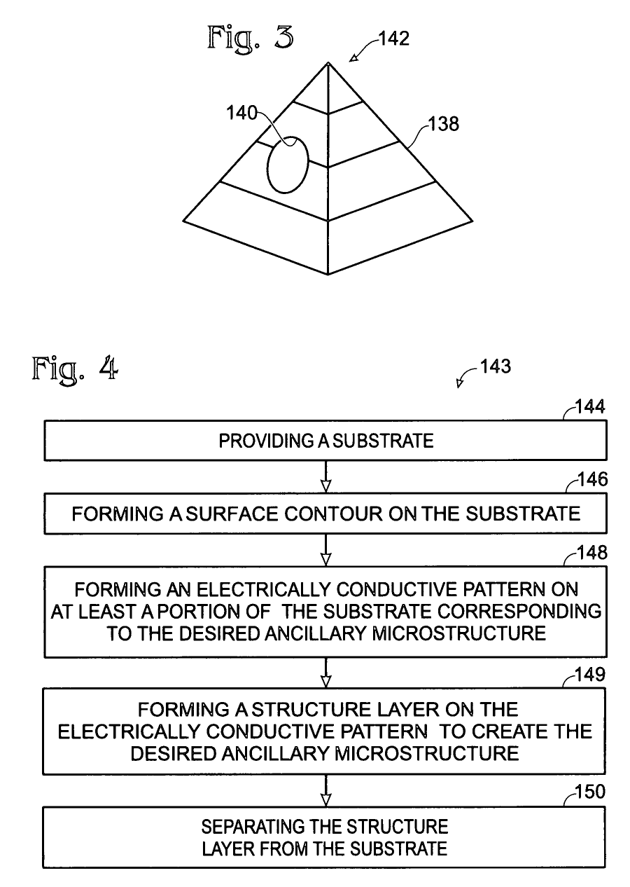

Ancillary microstructures may be prepared similarly. The microfabrication process of ancillary microstructures is depicted in flowchart 143 of FIG. 4, and includes providing a substrate at 144; forming a surface contour on the substrate at 146; forming an electrically conductive pattern on the substrate corresponding to the desired ancillary microstructure at 148; forming a structure layer on the electrically conductive pattern to create the desired ancillary microstructure at 149; and separating the structure layer from the substrate at 150.

The provided substrate may include any surface substance material suitable for the conditions experienced during microfabrication. The substrate may include a semiconductor material, such as silicon, a nonconductive material (such as glass), a metal (such as stainless steel or aluminum), a premolded or stamped polymer, such as a plastic or silicone polymer, among others. Where the substrate is a silicon substrate, it may be dry etched by ion milling or with a number of reactive gasses such as SF₆, gas-assisted FIB, wet etched with isotropic etch compositions such as mixtures of hydrofluoric-nitric-acetic, or etched with an anisotropic etch such as tetramethyl ammonium hydroxide (TMAH) in water or potassium hydroxide (KOH) in water, among others. Depending upon the composition of the substrate, alternative etching formulations may be used, including wet etching, dry etching, or various combinations of the above etching techniques, among others, in order to provide any of a variety of possible substrate starting shapes.

For-the purposes of illustration, the particular contour of the substrate depicted herein is a pyramidal contour. However the particular size, shape, and configuration of the contour to be etched may be varied in order to accommodate the desired geometry of the ancillary microstructure to be fabricated. Additionally, any other method of forming the desired contour, such as the use of chemical etching, stamping, imprinting, physical machining, reactive ion etching (RIE), ion milling, focused ion beam (FIB) etching, or photolithographic techniques, among others, is a suitable method of forming the desired contour in the selected substrate.

Once the desired contour has been formed, an electrically conductive pattern is formed on the substrate, where the pattern is configured to correspond to the desired ancillary microstructure. The electrically conductive pattern may be formed in any of a variety of manners, for example by depositing an electrically conductive material and then masking it with a non-conductive material; by applying an electrically conductive material and etching the material to create the desired pattern; or any other suitable method.

In one embodiment, the electrically conductive pattern is formed via application of an electrically conductive seed layer. The seed layer may be formed on, above, or adjacent to the surface of the substrate. Any sufficiently conductive material may be used to form the seed layer. In one embodiment, the seed layer may include one or more metals. For example, the seed layer may include a thin layer of chrome, tungsten, stainless steel, tantalum, gold or conductive polymer, and may be formed by sputtering or other conventional deposition techniques. The seed layer may be a single layer, for example a tungsten single layer, or multiple layers, such as stainless steel over chrome (Cr/SS), gold over tantalum (Ta/Au), or gold over molybdenum over titanium (Ti/Mo/Au). The thickness of the seed layer may range from about 500 Ångstroms to about 20,000 Ångstroms.

After deposition of the seed layer, deposition of a nonconductive layer on or adjacent to the seed layer may be used to create the electrically conductive pattern, and thereby help shape the subsequent structure layer. The nonconductive pattern may be formed by such methods as: using a photolithographic mask on the nonconductive layer followed by etching, formed through the use of a shadow mask without etching, formed by lift-off where the mask is placed first, or formed by gas-assisted ion beam deposition without the need for a mask. Suitable materials for the nonconductive pattern include silicon carbide, photoresists, polymers such as SU-8 (a resin available from Microchem Corp., Newton Mass.) silicon nitrides, silicon oxides, and silicon oxy-nitrides, among others. The thickness of the nonconductive pattern may range from about 500 Ångstroms to about 50,000 Ångstroms.

Application of a nonconductive substance may be performed concurrent with formation of the nonconductive pattern. Alternatively, the nonconductive substance may be applied first, and the pattern formed subsequently in a separate process. Patterning may be carried out both before and after deposition where a lift-off technique is used, or concurrent with masking if a shadow mask is used. Application of the nonconductive substance on the substrate may include application onto one or more intervening layers.

The structure layer may be prepared by application of an appropriate electrically conductive material above, adjacent to or upon the electrically conductive pattern. The structure layer may be applied onto one or more intervening layers. A subsequent electrically conductive material used to form the structure layer may be distinct from the material used for the seed layer. The structure layer may include any of a variety of conductive materials depending, at least in part, on the intended use of the structure, and may include one or more distinct applied layers. For example, for use in medical applications, the structure layer may be made of palladium, silver, gold, or alloys thereof, among others. The material(s) forming the structure layer may be selected for one or more of mechanical strength, biocompatibility, ability to be easily and uniformly electroplated into thick films, chemical stability (e.g. corrosion resistance), and ability to be selectively etched away from other materials used in the fabrication process. For example, nickel may be used for forming the seed layer. Silver may be used for forming all or part of the structure layer, as silver may be selectively etched from nickel using a solution of nitric acid and hydrogen peroxide. Silver has particular utility as a structure layer because it has high mechanical strength, is biocompatible, and can be plated to a relatively thick film.

After the structure layer has been formed, it may be separated from the substrate. This may be done by physically peeling the structure layer from the substrate, separating the structure layer via an etched sacrificial layer, or separating the structure layer with the aid of ultrasonic agitation.

In an alternative fabrication method, one or more release layers, either individually or in combination may be utilized to facilitate separation of the structure layer. By employing one or more release layers, the resulting structure layer may be more easily separated from the substrate. A release layer may be any material that is compatible with the formation process used to construct the structure layer. In one aspect of the disclosed method, the release layer is a temperature-sensitive layer that includes one or more materials selected to have a lower melting temperature than the surrounding materials. If the temperature-sensitive layer is a conductive material it may also be plated, thereby ensuring complete coverage of conductive parts. In this aspect, upon formation of the structure layer, heating the substrate may soften or melt the release layer and thereby allow separation of the two or more layers being held together by the release layer. The application of a release layer permits more variation between the materials of the seed layer and the plated structure layer, either by using the release layer itself as a shape modifier for the resulting plated structure layer, or by permitting additional layers to be added to the structure during fabrication due to the additional structural cohesion provided by the release layer. These factors all contribute to the fabrication of more complicated structures.

As depicted in the exemplary fabrication methods provided below, a release layer may be applied above the seed layer, on the seed layer, as the seed layer, or (in some circumstances) below the seed layer. For example, a release layer may be electroplated or otherwise formed on the exposed upper surface of the substrate, but not deposited on a previously applied non-conductive pattern. The release layer may therefore be formed in a pattern that substantially covers the portions of the seed layer that are not covered by the non-conductive layer. The release layer may substantially cover the seed layer so that when a structure layer is formed on or above the release layer, heating of the release layer is sufficient to allow the releasable structure layer to detach from the substrate without significant force.

In addition to the above method, a variety of alternative fabrication strategies may be used to prepare the disclosed ancillary microstructures. For example, rather than employing a patterned non-conductive layer, a patterned electrically-conductive seed layer may be applied to a uniform and non-conductive substrate, for example by deposition and etch, lift off, shadow mask, etc. The release layer may then be applied to the patterned seed layer prior to formation of a structure layer. This process may produce a smoother structure without the need of a discrete non-conductive pattern.

In another variation, the seed layer itself may function as a release layer. For example, a uniform and non-conductive layer may be covered by application of a uniform and conductive release layer, for example, by sputtering. The release layer may then be etched using photolithographic techniques to create a desired pattern by exposing the non-conductive material beneath the release layer, for example by lift off, shadow mask, etc. A structure layer may then be applied as discussed above. This process may also yield smoother microstructures without the necessity of a countersunk patch due to the presence of a discrete nonconductive pattern.

Where the fabrication process is directed to nesting microstructures, a release layer may be applied later in the process, such as after a first structural layer is deposited, but before the second structural layer is applied. This may result in discrete microstructures that may be separated from the substrate and/or from each other, but that nonetheless will nest within one another, such that they may be self-aligning.

Using yet another fabrication strategy, a release layer may be applied prior to the application of a patterned non-conductive layer. In this manner when the release layer is heated, the combined structure layer and non-conductive layer may be released, at which point the non-conductive pattern can be removed via, for example, etching methods. Alternatively, the non-conductive pattern may be permanently incorporated in the resulting microstructure.

Where the fabrication process includes application of a release layer, the release layer may be applied to different layers, and at different points and at multiple points in aggregate in the fabrication process, in order to create the desired microstructures. The preparation of the microstructures described in the following examples may be modified to include the application of one or more release structures, as described above.

Through the careful selection of an appropriate fabrication strategy, substrate contouring, formation of the nonconductive pattern, and formation of the structure layer, the present method permits the fabrication of a variety of microstructures, including more reliable microneedles, and microneedles with ancillary structures such as flap valves, microsprings, ridges, contact switches, barbed needles, and diverting structures.

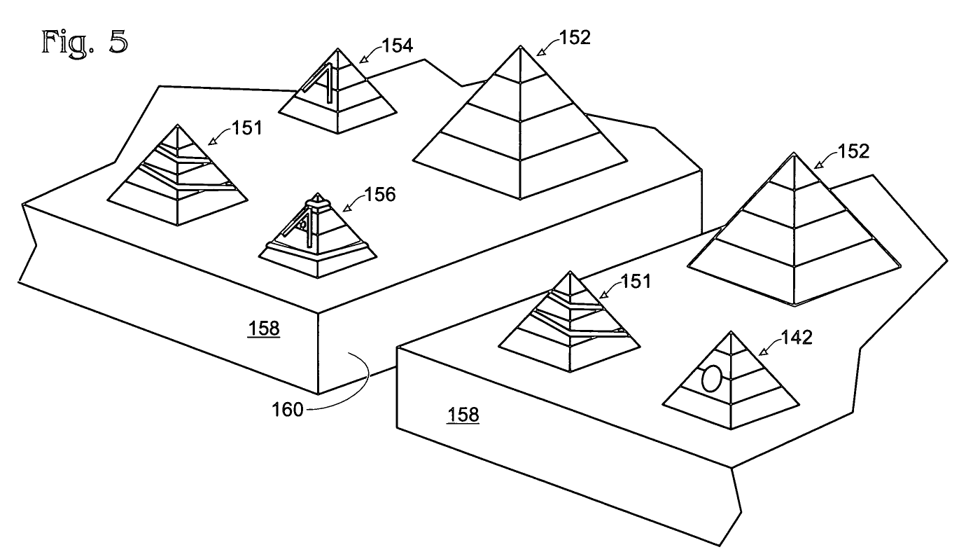

A variety of exemplary ancillary microstructures are shown in FIG. 5, in conjunction with associated microneedles. In addition to microneedle 142, the array of FIG. 5 includes microsprings 151, diverting structures 152, a valved microneedle 154, and a valved and ridged microneedle 156. The microneedles and their ancillary microstructures are depicted as being formed on a substrate 158 that incorporates a gap or channel 160. As discussed above, the pyramidal shape of the depicted microstructures is intended to be illustrative, and a variety of geometries and configurations may be prepared using the microfabrication methods disclosed herein. For example, although the exemplary microstructures are depicted as hollow structures, it should be appreciated that solid microstructures may be similarly prepared, and offer alternative or additional advantages over hollow microstructures.

In one embodiment, the fabricated microstructure is a microspring (see, for example, microspring 151 in FIG. 5). A microspring is a microstructure that is configured to deform under pressure, and thereby supply a resistive and resilient force. The presence of microsprings in a microneedle array may provide the user with force feedback, or tactile feedback regarding the pressure being applied against the membrane or surface to be pierced. In addition, the presence of microsprings associated with one or more microneedles may assist the penetration of the membrane and/or surface by the microneedles by stretching the membrane and/or surface and keeping the membrane and/or surface taut. This may be particularly advantages where the microneedle(s) are used to penetrate skin.

The microspring may exhibit resilience under deformation by incorporating any of a number of structures. For example, the microspring may incorporate a spiral aperture to confer a spring-like resilience. Alternatively, the microspring may incorporate a cantilevered structure, a semi-elliptical spring structure, or other flexible structure in order to provide the desired resilience. The microspring structure may incorporate any of a variety of tip geometries, such as a sharp tip, a blunt tip, and a rounded tip, among others, and similar structures may exhibit different tip geometries within a single microstructure array.

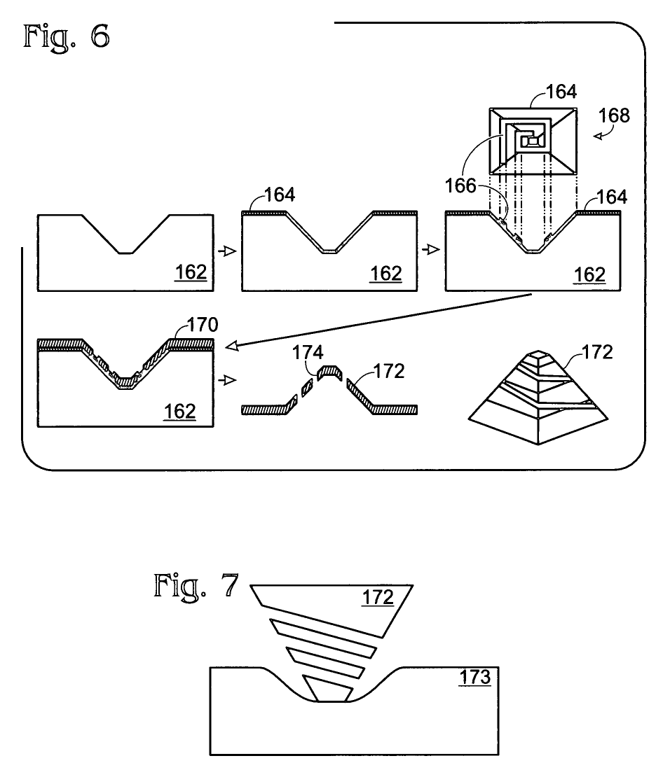

A representative fabrication of an ancillary microspring is depicted schematically in FIG. 6, beginning with the preparation of a substrate 162 that is anisotropically etched to form the desired concave shape. A seed layer 164 is then applied via sputter deposition, with the subsequent deposition and patterning of a non-conductive layer 166. The pattern on non-conductive layer 166 is configured as a spiral or other appropriate geometry to yield a final microstructure that exhibits a resilient resistance to deformation. A top view of the seed layer 164 and the deposited non-conductive layer 166 is shown at 168.

The structure layer 170 is electroplated onto the combined substrate 162 and seed layer 164, but not on the deposited nonconductive pattern 166, and follows the shape of the anisotropic concavity in the substrate 162. The electroplated microspring 172 may then be removed from the substrate as discussed above. The resulting patterned aperture 174 provides the structure with the desired springlike mechanical flexibility.

As shown in FIG. 7, when microspring 172 is pressed against a membrane 173, such as skin, it will apply a gentle stretching force to the membrane 173, making the membrane 173 tauter in the immediate area of the microspring 172. This may serve to facilitate insertion of an associated microneedle into the membrane 173.

In an alternative embodiment, the fabricated microstructure is a microvalve, for example a microvalve disposed on a microneedle (see, for example, valved microneedle 154 in FIG. 5). As used herein, a microvalve is a structure disposed on a hollow microneedle that is configured to be moved from a first to a second position. In one embodiment of the microvalve, the microvalve is configured to move from an opened to a closed position, or from a closed to an opened position. The valve may move in a reversible, or in an irreversible manner. The microvalve may therefore be used to initiate a flow of material from or to a microneedle, or to halt a flow of material from or to a microneedle or associated sample reservoir. The microvalve may be resilient, in the sense that an applied force may move the microvalve from an opened to closed position or vice versa. For example, the microvalve may open when subjected to internal pressure, and then return to a closed configuration when that pressure is removed. Alternatively, the microvalve may open when pressed against a membrane during penetration by an associated microneedle, and then return to a closed configuration when the microneedle is removed from the membrane.

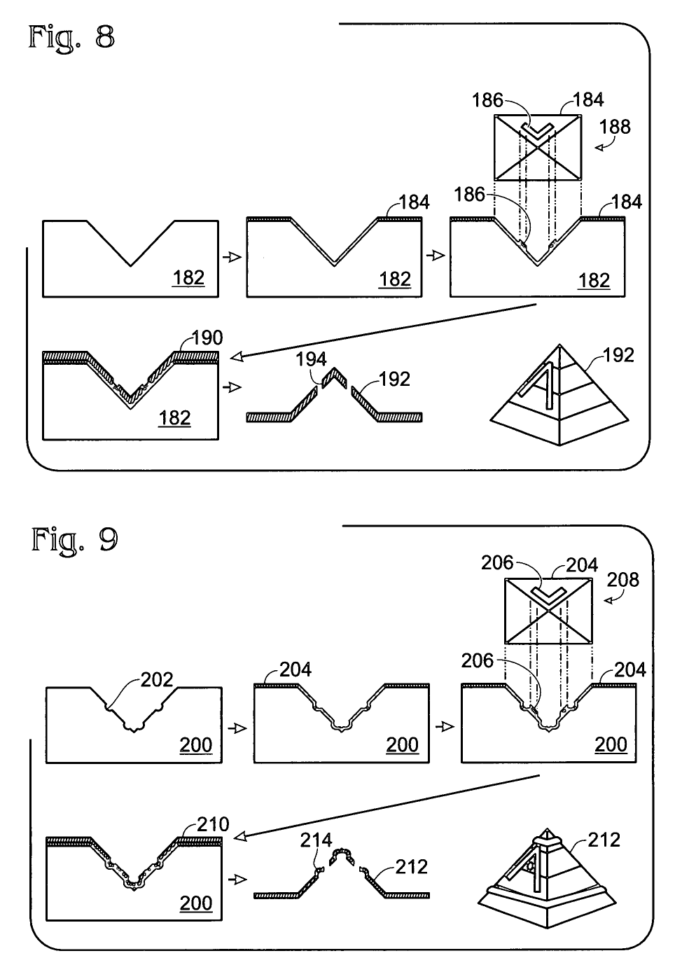

A representative fabrication of a valved microneedle is depicted schematically in FIG. 8, beginning with the preparation of a substrate 182 that is anisotropically etched to form the desired concave shape. A seed layer 184 is then applied via sputter deposition, with the subsequent deposition and patterning of a non-conductive layer 186. The pattern of non-conductive layer 186 is configured in the shape of a chevron, however any other appropriate geometry that will create valve capable of opening in a reversible manner is a suitable geometry for the microvalves disclosed herein. A top view of the seed layer 184 and the deposited non-conductive layer 186 is shown at 188.

The structure layer 190 is applied to the substrate 182 and seed layer 184, but not on the deposited nonconductive pattern 186, and generally follows the shape of the anisotropic concavity formed in the substrate 182. For example, the structure layer 190 may be electroplated onto the substrate 182. The microstructure 192 may then be removed from the substrate as discussed above. The resulting patterned aperture 194 provides the structure 192 with a chevron-shaped flexible valve, configured to open under pressure. Where microstructure 192 is a microneedle, the valve may be useful in regulating the delivery or collection of a substance, such as a medicament, by the associated microneedle.

In still another embodiment, the fabricated microstructure is a ridged microstructure (see, for example, ridged microneedle 156 in FIG. 5). A pattern of ridges may be fabricated onto a microstructure, such as a microneedle, in order to make it more difficult to insert the microneedle into a membrane. Alternatively, or in addition, the ridges may be configured to make it more difficult to dislodge or withdraw the microneedle from the membrane. The ridges or protrusions may be configured to ensure that the microneedle, or other microstructure, remains properly anchored to the desired membrane or other surface during use.

A representative fabrication of a ridged structure is depicted schematically in FIG. 9, beginning with the preparation of a substrate 200 that is anisotropically etched to form the desired concave shape. In this embodiment, rather than a smooth contour, the substrate 200 has been shaped to incorporate additional depressions, or channels 202. A seed layer 204 is then applied to the shaped contour via sputter deposition, with the subsequent deposition and patterning of a non-conductive layer 206. The pattern of non-conductive layer 206 may be configured to produce a microspring structure, a valve structure, a microneedle structure, or any other suitable structure. As shown in FIG. 9, layer 206 is configured to yield a chevron-shaped microvalve. A top view of the seed layer 204 and the deposited non-conductive layer 206 is shown at 208.

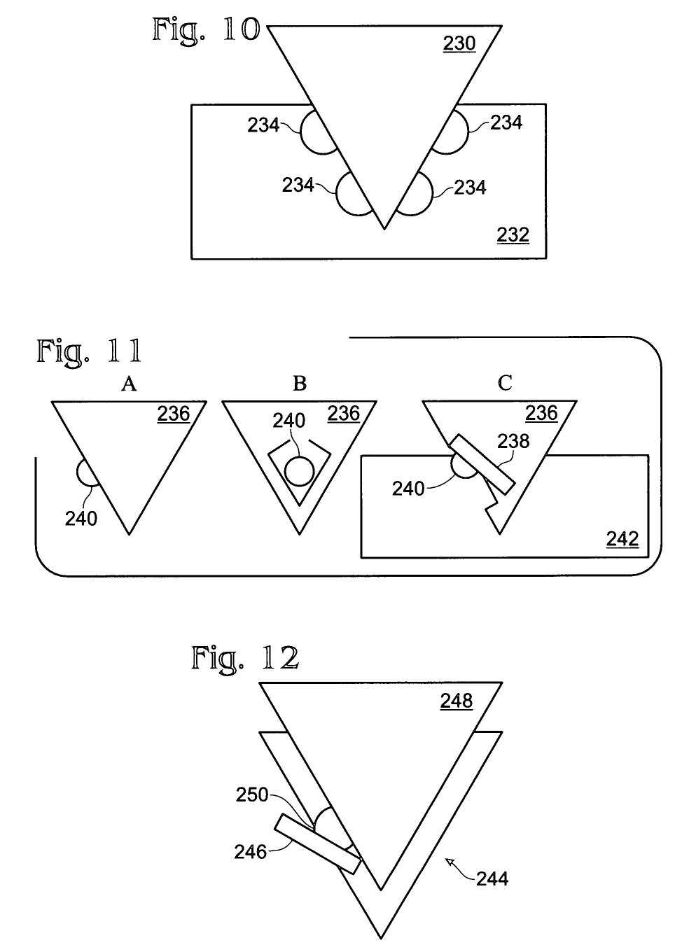

The structure layer 210 is electroplated onto the substrate 200 and seed layer 204, but not on the deposited nonconductive pattern 206, and additionally matches the shape of the contoured concavity in the substrate 200. The electroplated microstructure 212, including the ridge structures 214, may then be removed from the substrate as discussed above. FIG. 10 depicts a ridged microneedle 230 anchored in a membrane 232 via ridges 234.

In yet another alternative embodiment, the ancillary microstructure may be a microneedle incorporating a flap valve as depicted schematically in FIG. 11. Microneedle 236 incorporates a valve structure 238, as discussed previously. In addition, by employing an appropriate contoured substrate, a ridge or button 240 on the valve functions to force the valve to open inwardly upon insertion into a membrane and/or surface 242. In addition to functioning as a valve for substance delivery and/or collection, flap valve 238 may be used to create an electrical contact, thereby providing a signal when the microvalve is forced open. Such a construction could be used to determine the precise time of entry of the microstructure into a membrane, and in conjunction with additional microneedles, could serve to record the time medicament was initially delivered to a user. Alternatively, the construction may be used to establish whether the chamber is in fact open, in order to verify whether the associated microneedle is delivering or collecting at all.

In another alternative embodiment, the ancillary microstructure may incorporate one or more microbarbs, as shown in FIG. 12. Microbarbed structure 244 includes a microflap structure 246, as discussed previously, but instead of being depressed inward, the microflap structure 246 may be urged outward by insertion of a complementary structure 248 having a, ridge or button 250 configured to force microflap structure 246 outward. The resulting microbarb structure 244 may help prevent the structure, or an associated microneedle or microneedle array, from being dislodged from a membrane or other surface.

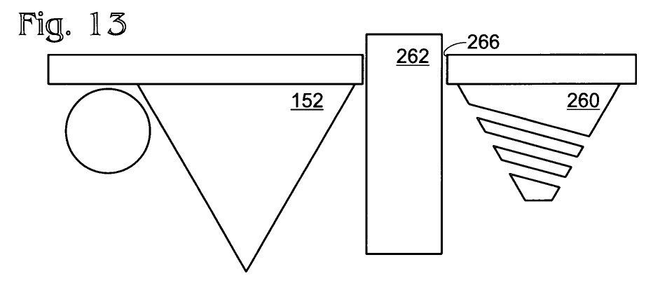

In yet another alternative embodiment, the ancillary microstructure may include a diverting structure that is associated with one or more adjacent microneedles or other microstructures. The presence of hair, interfering structure, or other matter on a target surface may prevent a microneedle from fully or properly penetrating that surface. As a result, the substance of interest may not be properly delivered and/or collected by the microneedle. The inclusion of one or more diverting structures associated with the microneedles may assist in diverting such interfering matter away from the microneedle structures. For example, as shown in FIG. 13, diverting structure 152 is shown in a section of a microstructure array that includes a microspring structure 260. Diverting structure 152 has diverted hair 262, and guided it into an aperture or channel 266 in the microstructure array, so that hair 262 does not interfere with the proper functioning of the microstructure array.

Although embodiments of the present disclosure have been shown and described with reference to the foregoing operational principles, it will be apparent to those skilled in the art that various changes in form and detail can be made without departing from the spirit and scope of the invention as defined in the following claims. Various configurations of microneedles, microvalves, microsprings, and other microstructures may be envisioned, as well as a variety of possible interactions between such microstructures. The present disclosure is intended to embrace all such alternatives, modifications and variances, including all novel and non-obvious combinations and subcombinations of the various elements, features, functions and/or properties disclosed herein.

What is claimed is:

1. A microstructure array, comprising
at least one microneedle; and
at least one ancillary microstructure on or adjacent to the microneedle configured to modulate the function of the microneedle.

2. The microstructure array of claim 1, wherein the ancillary microstructure is configured to facilitate insertion of the at least one microneedle into a target surface.

3. The microstructure array of claim 2, wherein the at least one ancillary structure is a microspring structure configured to exert a force on the target surface.

4. The microstructure array of claim 3, wherein the microspring structure is configured to supply a resistive and resilient force under deformation.

5. The microstructure array of claim 3, wherein the microspring structure includes a hollow projection having a surface that defines a substantially spiral aperture.

6. The microstructure array of claim 3, wherein the microspring structure is configured to be reversibly deformed.

7. The microstructure array of claim 1, wherein the at least one ancillary structure includes a ridge, button, barb or combinations thereof associated with the at least one microneedle, and wherein the ridge, button, barb or combinations thereof is configured to help retain the at least one microneedle within a target surface.

8. The microstructure array of claim 1, further comprising:
at least one other microneedle; and
at least one other ancillary microstructure on or adjacent to the at least one other microneedle, the at least one other ancillary microstructure being different from the at least one ancillary structure associated with the at least one microneedle.

9. The microstructure array of claim 1, wherein the at least one ancillary structure includes a reversible microvalve.

10. The microstructure array of claim 9, where the microvalve is configured to be reversibly opened.

11. The microstructure array of claim 1, wherein the at least one ancillary structure is a diverting structure that is configured to divert hair or particles away from the at least one microneedle.

12. The microstructure array of claim 1, wherein the ancillary microstructure is configured to supply a resistive and resilient force under deformation.

13. The microstructure array of claim 12, wherein the ancillary microstructure includes a hollow projection.

14. The microstructure array of claim 13, wherein a surface of the hollow projection defines a substantially spiral patterned aperture.

15. The microstructure array of claim 12, wherein the ancillary microstructure is configured to exert a resistance to deformation when pressed against a target surface.

16. The microstructure array of claim 15, wherein the ancillary microstructure is configured such that when pressed against a deformable surface, the ancillary microstructure helps to tauten the deformable surface.

17. (canceled)

18. A method of fabricating an ancillary microstructure on or adjacent to a microneedle, comprising:
providing a substrate;
forming a surface contour on the substrate;
forming an electrically conductive pattern on at least a portion of the substrate corresponding to the ancillary microstructure;
forming a structure layer on the electrically conductive pattern to create the ancillary microstructure configured to modulate the function of the microneedle; and
separating the structure layer from the substrate.

19. The method of claim 18, wherein the ancillary microstructure is a microspring that is configured to be resiliently and reversibly depressed.

20. The method of claim 19, wherein the nonconductive pattern incorporates at least a portion of a substantially spiral pattern.

21. The method of claim 18, further comprising forming a release layer configured to facilitate separating the structure layer from the substrate.

22. The method of claim 18, wherein the ancillary structure is selected from a barb, a microvalve, a diverting structure, a rigid microstructure, or a flap valve.
